(19) United States
(12) Patent Application Publication
(10) Pub. No.: US 2012/0253326 A1
(43) Pub. Date: Oct. 4, 2012

(54) Articulation of Laparoscopic Instrument

(75) Inventor: Gennady Kleyman, Brooklyn, NY (US)

(73) Assignee: Tyco Healthcare Group LP, Mansfield, MA (US)

(21) Appl. No.: 13/412,065

(22) Filed: Mar. 5, 2012

Related U.S. Application Data
(60) Provisional application No. 61/468,871, filed on Mar. 29, 2011.

(51) Int. Cl. A61B 17/00

(52) U.S. Cl. 606/1

(57) ABSTRACT

A surgical articulation apparatus and method for articulation of laparoscopic instruments is disclosed. The surgical articulation apparatus includes a housing, a tubular member extending distally from the housing, a spherical member attached to a proximal end of the housing, a ring member disposed about the spherical member and a cable extending from the tubular member through the housing to the ring member. The housing, tubular member and spherical member each include lumens therethrough for the reception of a flexible surgical instrument. The ring member is rotatable about the spherical member. The cable is attached to the tubular member and disposed in mechanical communication with the ring member such that the tubular member is manipulated by cable upon rotation of the ring member about the spherical member.

### CROSS REFERENCE TO RELATED APPLICATIONS

The present application claims the benefit of and priority to U.S. Provisional Application Ser. No. 61/468,871, filed on Mar. 29, 2011, the entire contents of which are incorporated herein by reference.

### BACKGROUND

1. Technical Field

The present disclosure relates generally to articulation of laparoscopic instruments. More particularly, the present disclosure relates to the use of a gimbal structure to allow for an easy and intuitive way of controlling and articulating the distal end of a surgical articulation apparatus.

2. Description of Related Art

Increasingly, many surgical procedures are performed through small incisions in the skin. As compared to the larger incisions typically required in traditional procedures, smaller incisions result in less trauma to the patient. By reducing the trauma to the patient, the time required for recovery is also reduced. Generally, the surgical procedures that are performed through small incisions in the skin are referred to as endoscopic. If the procedure is performed on the patient's abdomen, the procedure is referred to as laparoscopic. Throughout the present disclosure, the term minimally invasive is to be understood as encompassing both endoscopic and laparoscopic procedures.

During a typical minimally invasive procedure, surgical objects, such as surgical access devices (e.g., trocar and cannula assemblies) or endoscopes, are inserted into the patient's body through an incision in tissue. A surgeon is required to manipulate a surgical articulation apparatus through the use of a series of knobs or other control mechanisms to perform a surgical operation. These knobs and control mechanisms can be both slow and cumbersome for the surgeon to use thereby prolonging the surgical operation and taking the surgeon's concentration away from the surgical operation.

Thus an easy and intuitive way of controlling and articulating the surgical articulation apparatus is desired.

### SUMMARY

A surgical articulation apparatus and method for articulation of laparoscopic instruments is disclosed. The surgical articulation apparatus provides for an easy and intuitive way of articulating a distal end of the surgical articulation apparatus through the use of a gimbal structure. The gimbal structure has a spherical member and a ring member disposed about the spherical member. The ring member is mechanically associated with the distal end of the surgical articulation apparatus by the use of a cable and is rotatable about the spherical member to move the cable proximally and distally relative to the surgical articulation apparatus. The cable is attached to the distal end of the surgical articulation apparatus and routed from the distal end of the surgical articulation apparatus to the gimbal structure. The cable is then looped through two sets of holes found on opposite sides of the ring member before returning to the distal end of the surgical articulation apparatus for further attachment. Upon rotation of the ring member about the spherical member a first end of the cable moves proximally while a second end of the cable moves distally thereby causing the distal end of the surgical articulation apparatus to bend. Thus the surgical articulation apparatus is easy and intuitive to use due to the relationship between the movement of the ring and the movement of the distal end of the surgical articulation apparatus where, for example, when the surgeon manipulates a side of the ring proximally, the distal end of the surgical articulation apparatus is bent toward the same side. Because of this, the surgeon no longer needs to determine which direction a control knob or control mechanism must be moved to get a desired effect and can instead concentrate solely on the procedure at hand.

Referring now to the structure of the surgical articulation apparatus, the surgical articulation apparatus includes a housing, the housing defining a housing lumen therethrough for the reception of a surgical instrument, a tubular member extending distally from the housing, the tubular member defining a flexible lumen therethrough for the reception of the flexible surgical instrument, a spherical member attached to a proximal end of the housing, the spherical member defining a sphere lumen therethrough for the reception of the flexible surgical instrument and defining a longitudinal axis, a ring member disposed about the spherical member and rotatable with respect to the spherical member and a cable attached to the tubular member and extending through the housing to the ring member. The cable is disposed in mechanical communication with the ring member such that the tubular member is articulated by the cable upon rotation of the ring member about the spherical member.

The cable may define two ends, each end being attached to a distal section of the tubular member. The ring member may include a plurality of openings therethrough and the cable may be inserted through at least one of the plurality of openings. The ring member may further include a plurality of flanges disposed on its surface where the flanges are actuatable to rotate the ring member about the spherical member. The surgical articulation apparatus may also include an outer shell disposed around the housing, the ring member and the spherical member. The outer shell includes a handle portion defining a plurality of slots thereon for the reception of the plurality of flanges therethrough. The plurality of slots may be dimensioned to allow at least one of the flanges to rotate about a first axis perpendicular to the longitudinal axis and another of the flanges to rotate about a second axis perpendicular to both the first axis and the longitudinal axis. The tubular member may also include a series of flexible elements.

A method of use is also disclosed. The method includes providing a surgical articulation apparatus including a housing, the housing defining a housing lumen therethrough for the reception of a flexible surgical instrument, a tubular member extending distally from the housing, the tubular member defining a flexible lumen therethrough for the reception of the flexible surgical instrument, a spherical member attached to a proximal end of the housing, the spherical member defining a sphere lumen therethrough for the reception of the flexible surgical instrument and defining a longitudinal axis, a ring member disposed about the spherical member and rotatable about the spherical member and a cable attached to the tubular member and extending through the housing to the ring member, the cable being disposed in mechanical communication with the ring member such that the tubular member is articulated by the cable upon rotation of the ring member about the spherical member.

The method further includes inserting the tubular member into a surgical site, inserting the flexible surgical instrument through the sphere lumen, housing lumen and flexible lumen and into the surgical site, rotating the ring member about a first axis to manipulate the tubular member in a first direction and rotating the ring member about a second axis to manipulate the tubular member in a second direction. The ring member may further include a plurality of flanges disposed on its surface and the step of rotating the ring member about the first axis may include manipulating a first of the plurality of flanges about the first axis, the first axis being perpendicular to the longitudinal axis of the sphere lumen. The step of rotating the ring member about the second axis may include manipulating a second of the plurality of the flanges about the second axis, the second axis being perpendicular to the longitudinal axis of the sphere lumen and perpendicular to the first axis. The plurality of flanges may define a generally cross shaped pattern with flanges on three of the four sides.

Rotation of the ring member translates a first end of the cable in one of a distal or proximal direction and a second end of the cable in the other of the distal or proximal direction to articulate the tubular member such that the ends move in opposed directions. The first and second ends of the cable may be attached to a distal section of the tubular member and the cable may be looped through at least one hole in the ring member. The cable may also be looped through two holes in each of a first and second sides of the ring member before returning to the distal section of the tubular member. A second cable may be attached to the distal section of the tubular member and looped through two holes in each of a third and fourth sides of the ring member before returning to the distal section of the tubular member. The tubular member may further include flexible links. Upon movement of the cable in a distal or proximal direction, the flexible links allow the tubular member to bend.

### BRIEF DESCRIPTION OF THE DRAWINGS

The accompanying drawings, which are incorporated in and constitute a part of this specification, illustrate embodiments of the presently disclosed surgical device, and together with a general description of the presently disclosed surgical device given above, and the detailed description of the embodiments given below, serve to explain the principles of the presently disclosed surgical device.

FIG. 1 is a perspective view of a surgical articulation apparatus in accordance with the present disclosure;

FIG. 2 is a side perspective view of the proximal portion of the surgical articulation apparatus of FIG. 1;

FIG. 3 is a rear perspective view of the surgical articulation apparatus of FIG. 2;

FIG. 4 is a top plan view of the surgical articulation apparatus of FIG. 1;

FIG. 5 is a partial cut-away view of the surgical articulation apparatus of FIG. 4;

FIG. 6 is an exploded view of the outer shell of the surgical articulation apparatus of FIG. 1;

FIG. 7 is an exploded view of the housing and gimbal structure of the surgical articulation apparatus of FIG. 1;

FIG. 8 is an exploded view of the tubular member of the surgical articulation apparatus of FIG. 1;

FIG. 8A is bottom perspective view of one of the flexible links of the tubular member of FIG. 8;

FIG. 8B is top perspective view of the flexible link of FIG. 8A;

FIG. 8C is bottom view taken along section line 8C-8C of the flexible link FIG. 8B;

FIG. 9 is a schematic view of the gimbal structure of the surgical articulation apparatus of FIG. 1, showing the routing of one of the cables;

FIG. 10 is a side perspective view of the housing and the gimbal structure of the surgical articulation apparatus of FIG. 1;

FIG. 11 is a rear view taken along section line 11-11 of FIG. 10 showing the routing of the cables;

FIG. 12 is a perspective view of the distal section of the tubular member of the surgical articulation apparatus of FIG. 1;

FIG. 13 is a rear view of the proximal portion the surgical articulation apparatus of FIG. 1 showing the cables in phantom;

FIG. 14 is a bottom view taken along section line 14-14 of FIG. 13 showing the looping of the cable;

FIG. 14A is a perspective view of the surgical articulation apparatus of FIG. 1 showing rotation of the ring member about the “Y” axis and corresponding movement of the distal section of the tubular member;

FIG. 15 is a top view taken along section line 15-15 of FIG. 14A showing the movement of the cables upon rotation of the ring member;

FIG. 16 is a rear perspective view of the surgical articulation apparatus of FIG. 14;

FIG. 17 is a perspective view of the surgical articulation apparatus of FIG. 1 showing rotation of the ring member about the “Z” axis and corresponding movement of the distal section of the tubular member; and

FIG. 18 is a section view along section 18-18 of FIG. 17 showing movement of the cables upon rotation of the ring member.

### DETAILED DESCRIPTION

Disclosed herein is a surgical articulation apparatus which provides for an easy and intuitive way of articulating a distal end of the surgical articulation apparatus through the use of a gimbal structure to allow for both fine and coarse articulation of a laparoscopic instrument during a surgical procedure.

Particular embodiments of the presently disclosed surgical articulation apparatus are described in detail with reference to the drawing figures wherein like reference numerals identify similar or identical elements. As used herein, the term “distal” refers to that portion which is farther from the user while the term “proximal” refers to that portion which is closer to the user or surgeon.

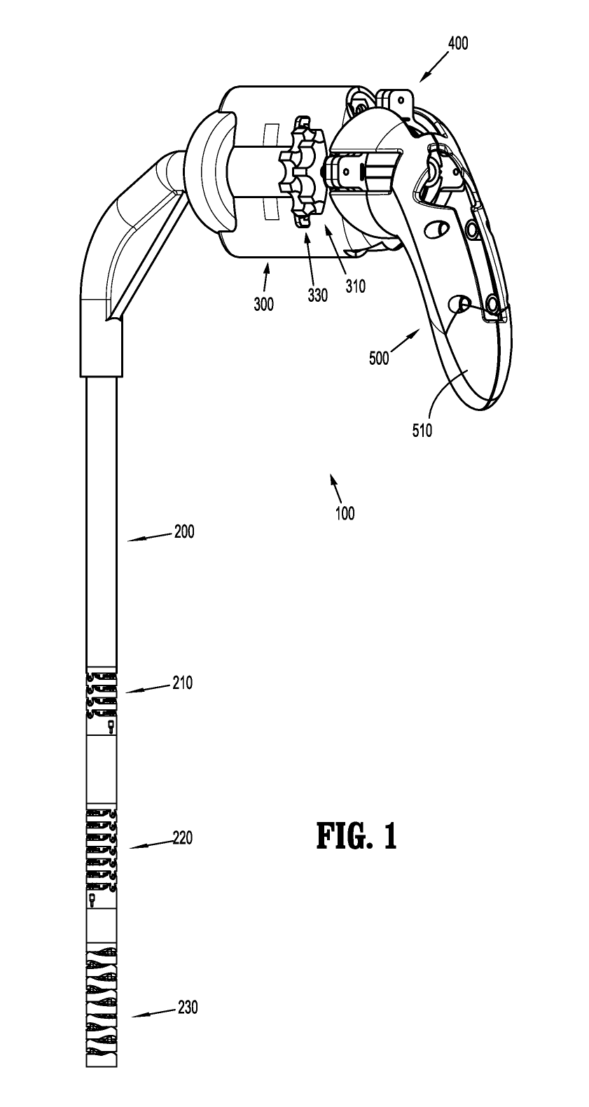

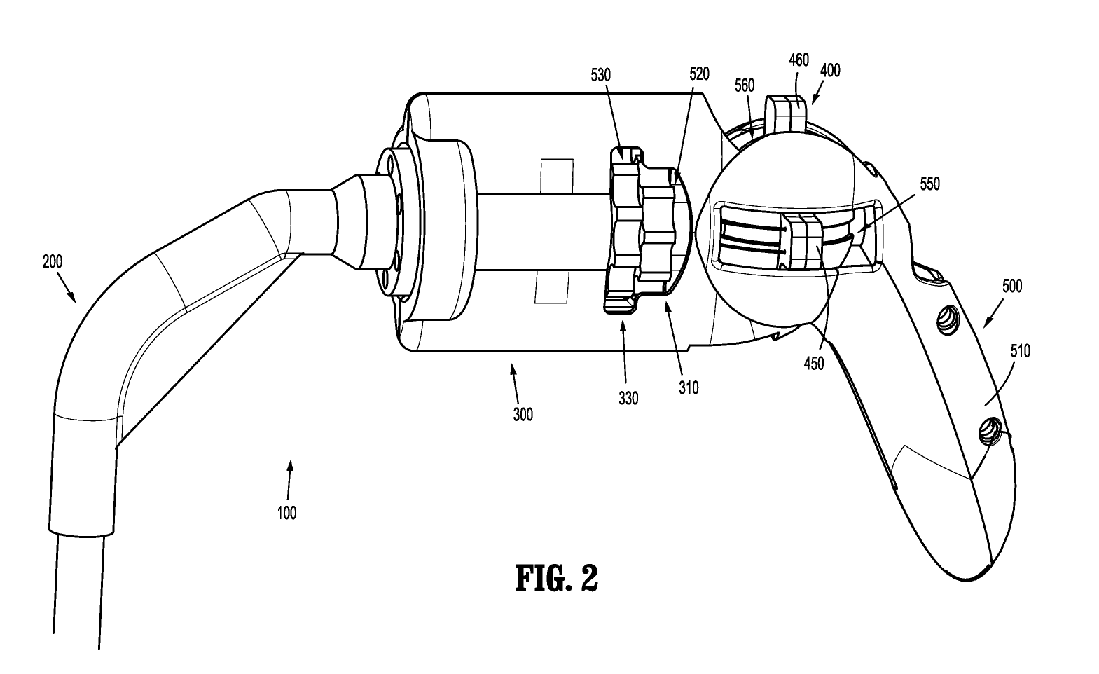

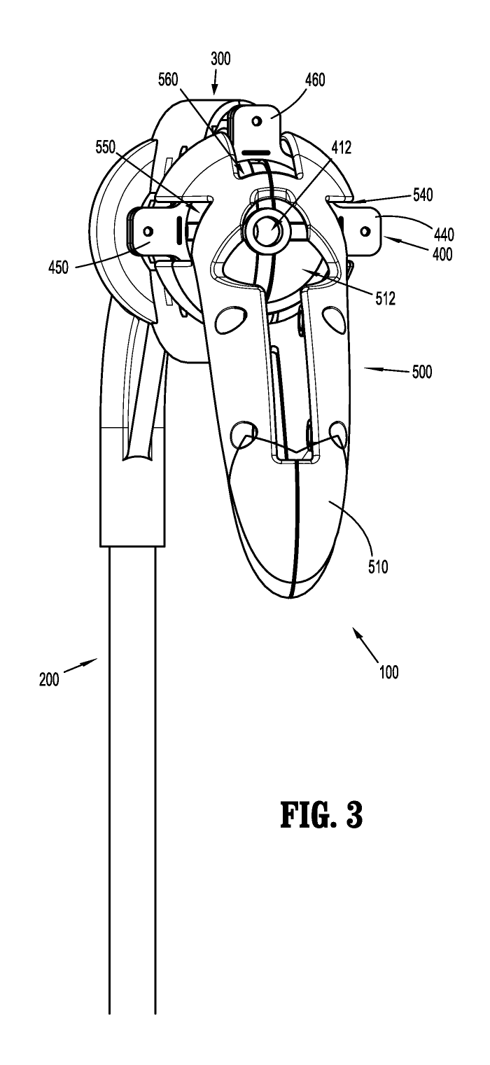

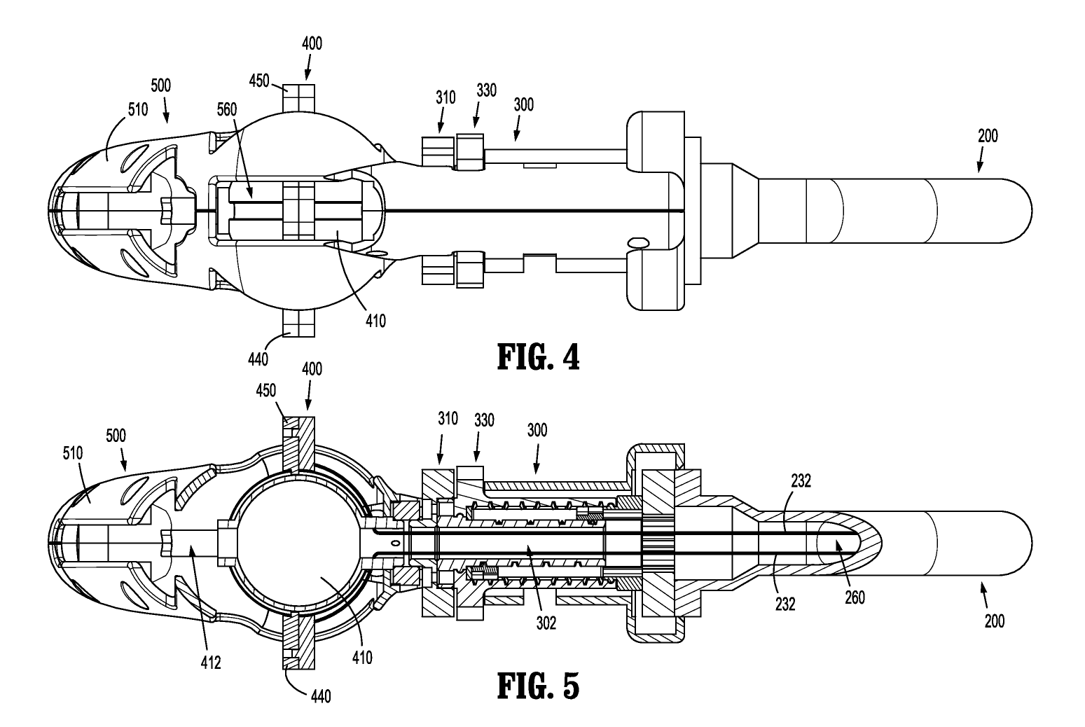

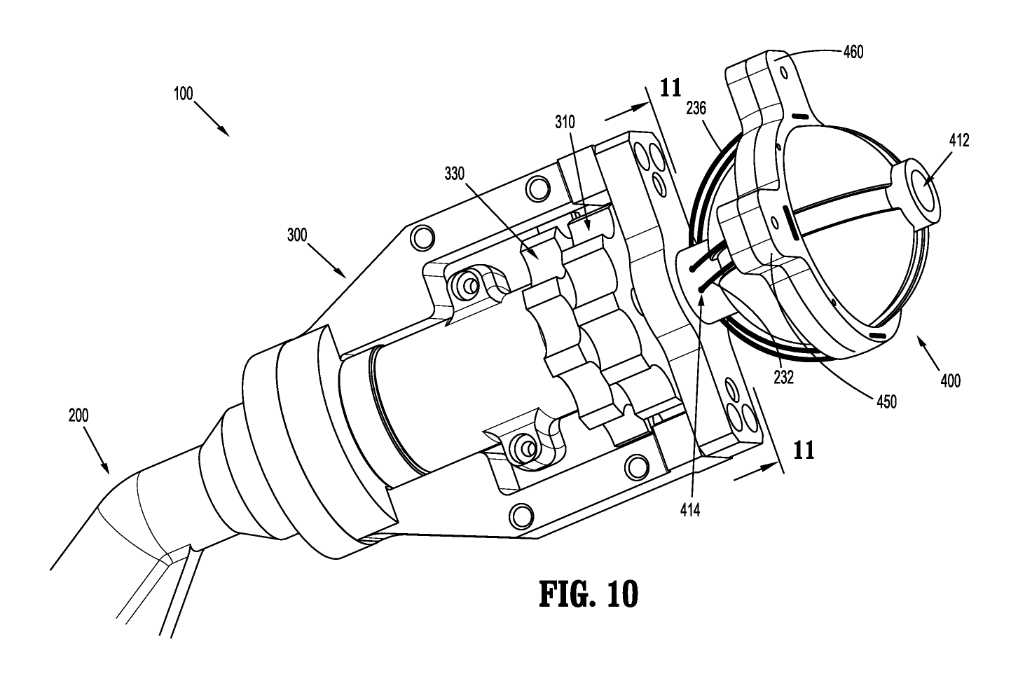

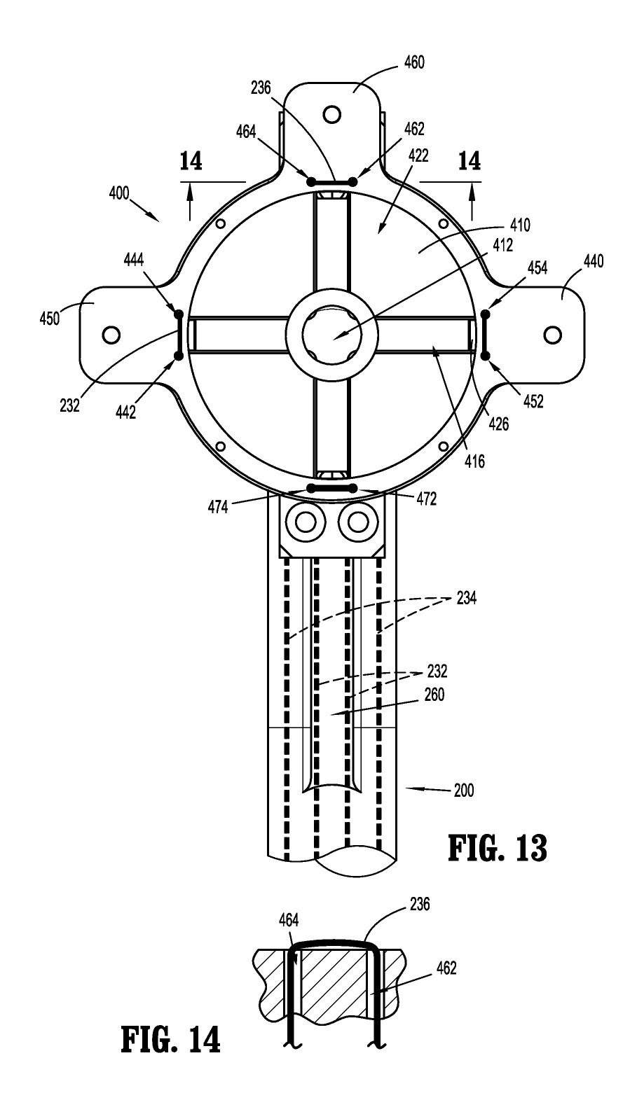

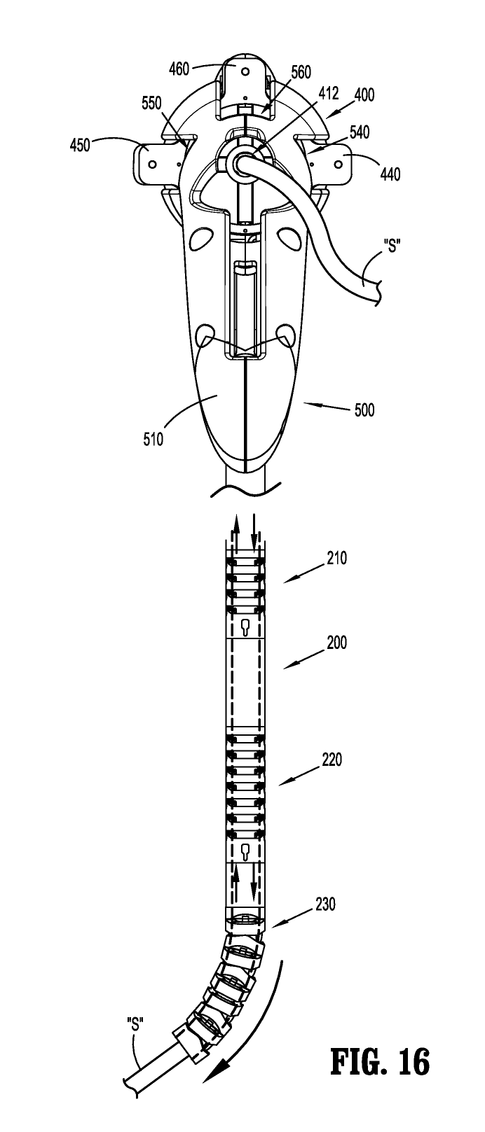

With reference to FIGS. 1-18 a surgical articulation apparatus for articulation of laparoscopic instruments 100 is disclosed including a tubular member 200, a housing 300 extending proximally from tubular member 200, a gimbal structure 400 attached to a proximal end of housing 300, and an outer shell 500 disposed around housing 300 and gimbal structure 400.

Referring now to FIGS. 1, 8, 8A, 8B, 8C and 12 tubular member 200 includes three articulation sections: a proximal section 210, a central section 220 and a distal section 230. Each section 210, 220 and 230 includes a respective pair of cables 212, 214, 222, 224 and 232, 236 extending therefrom to housing 300 and in the case of cables 232 and 236 through housing 300 and to gimbal structure 400.

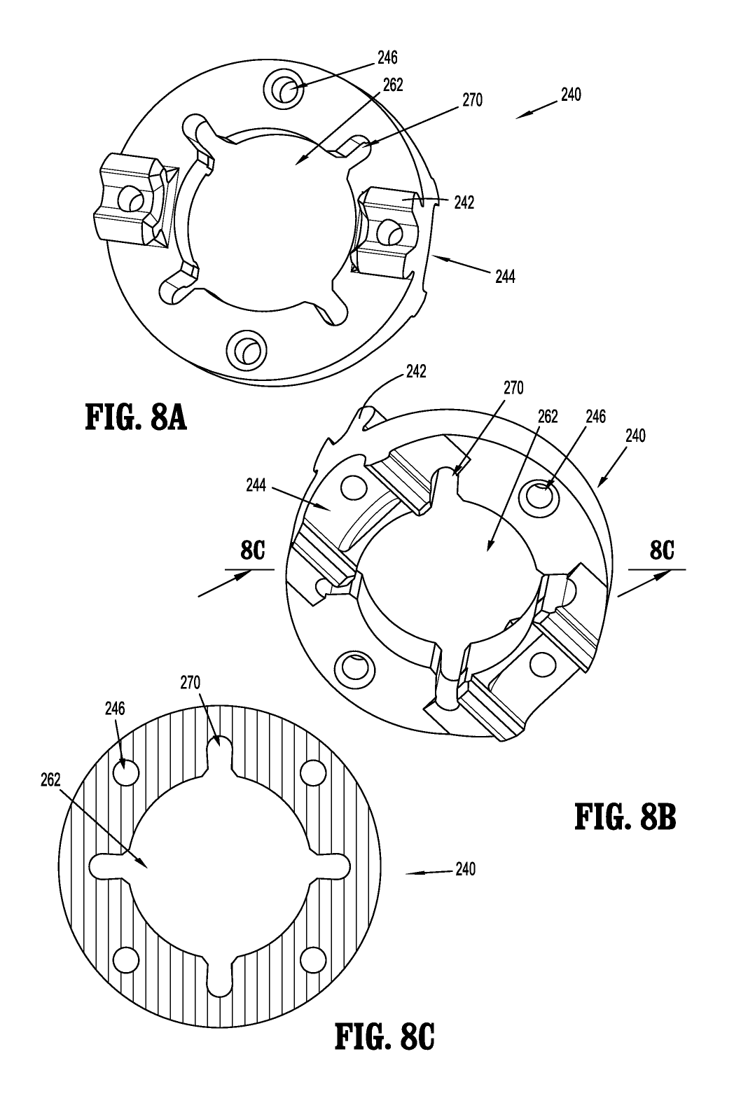

Proximal section 210 and central section 220 each include flexible links 240 to allow tubular member 200 to bend or flex. As best seen in FIGS. 8, 8A, 8B and 8C, each link 240 includes a set of nubs 242 and a set of recesses 244 which are substantially aligned with nubs 242. Recesses 244 are dimensioned for the reception of nubs 242 of an adjacent link 240. Each link 240 is rotatable with respect to adjacent links 240 to bend corresponding section 210 or 220. It is contemplated that flexible links 240 may be configured for bending in a single direction transverse to the longitudinal axis of tubular member 200, as seen in proximal and central sections 210, and 220 or that flexible links 240 may be configured for bending in multiple directions as will be describe with respect to distal section 230. Flexible links 240 also include guide lumens 246 for the reception of cables 212, 214, 222 and 224 therethrough and include channels 270 for the reception of cables 232 and 236 therethrough.

Referring now to FIGS. 8, 12, 14A, 16 and 17, distal section 230 includes articulating links 250 which are similar to flexible links 240 except that articulating links 250 include nubs 252 which are not aligned with recesses 254. Instead nubs 252 are transversely positioned with respect to recesses 254 such that adjacent articulating links 250 are alternately positioned to allow distal section 250 to bend in multiple directions. Articulating links 250 include lumens 256 which are substantially aligned with channels 270 for the reception of cables 232 and 236 therethrough.

Tubular member 200 further defines a flexible lumen 260 extending longitudinally therethrough including openings 262 in flexible links 240 and articulating links 250 for the reception of a flexible surgical instrument “S”. It is contemplated that a greater or fewer number of cables may be used. It is also contemplated that there may be a greater of fewer number of articulation sections.

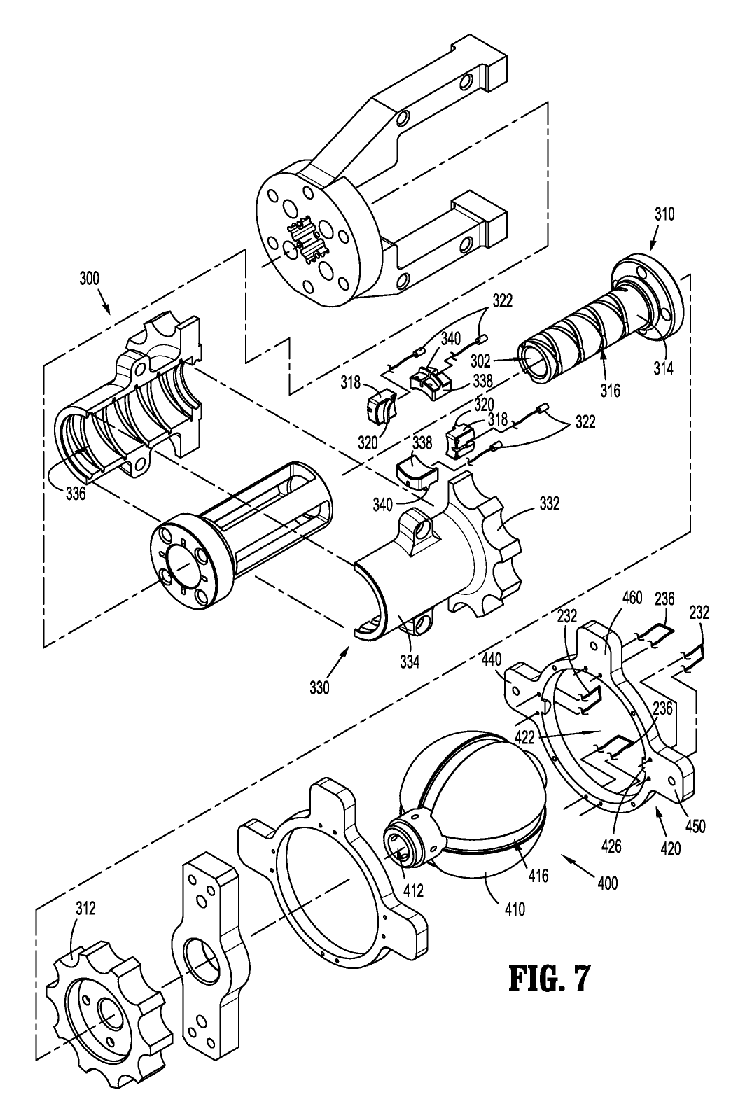

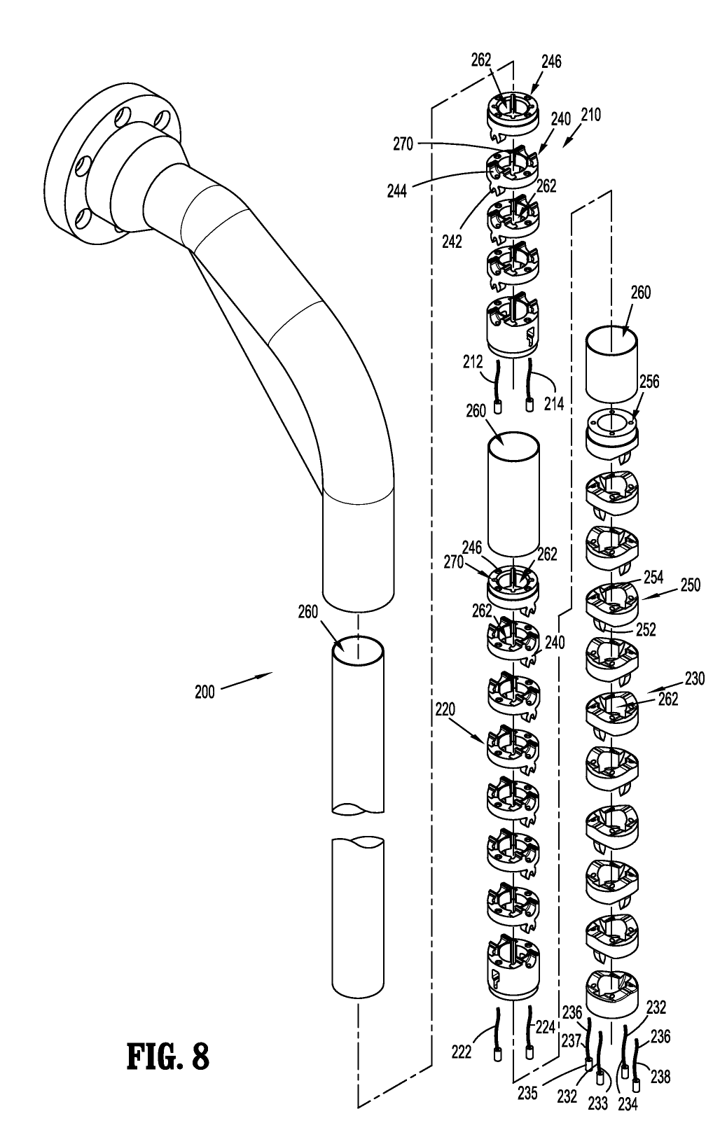

Referring now to FIGS. 6 and 7, housing 300 includes a housing lumen 302 extending therethrough for the reception of flexible surgical instrument “S” and two rotatable control members 310 and 330 disposed around housing lumen 302. The distal end of housing lumen 302 is generally aligned with the proximal end of flexible lumen 260 to allow flexible surgical instrument “S” to pass through housing lumen 302 and into flexible lumen 260. Control member 310 includes a knob 312, an elongate portion 314, and a pair of sliding cable mounts 318. Elongate portion 314 includes a series of grooves 316 defined on an outer surface and sliding cable mounts 318 include ridged portions 320 disposed on an inner surface and adapted to slide along grooves 316 upon rotation of knob 312. Sliding cable mounts 318 are adapted to receive cables 212 and 214 and travel along grooved portions 312 to increase or decrease the tension on cables 212 and 214 to manipulate proximal section 210 of tubular member 200. Cables 212 and 214 are secured to sliding cable mounts 318 by the use of a ferrule 322 or may alternatively be secured to sliding cable mounts 318 by welding other means as known in the art.

Control member 330 includes a knob 332, an elongate portion 334, and a pair of sliding cable mounts 338. Elongate portion 334 includes a series of grooves 336 defined on an inner surface and sliding cable mounts 338 include ridged portions 340 disposed on an outer surface and adapted to slide along grooves 336 upon rotation of knob 332. Sliding cable mounts 338 are adapted to receive cables 222 and 224 and travel along grooved portions 332 to increase or decrease the tension on cables 222 and 224 to manipulate central section 220 of tubular member 200. Cables 222 and 224 are secured to sliding cable mounts 338 by the use of a ferrule 322 or may alternatively be secured to sliding cable mounts 338 by welding other means as known in the art.

Each control member 310 and 330 may be actuated independently to articulate proximal section 210 and central section 220 independently. It is further contemplated that additional control members may be provided such that additional articulation sections may be manipulated or alternatively such that more than one control member may be actuated for articulating a single section where each control member articulates the single section in a different direction.

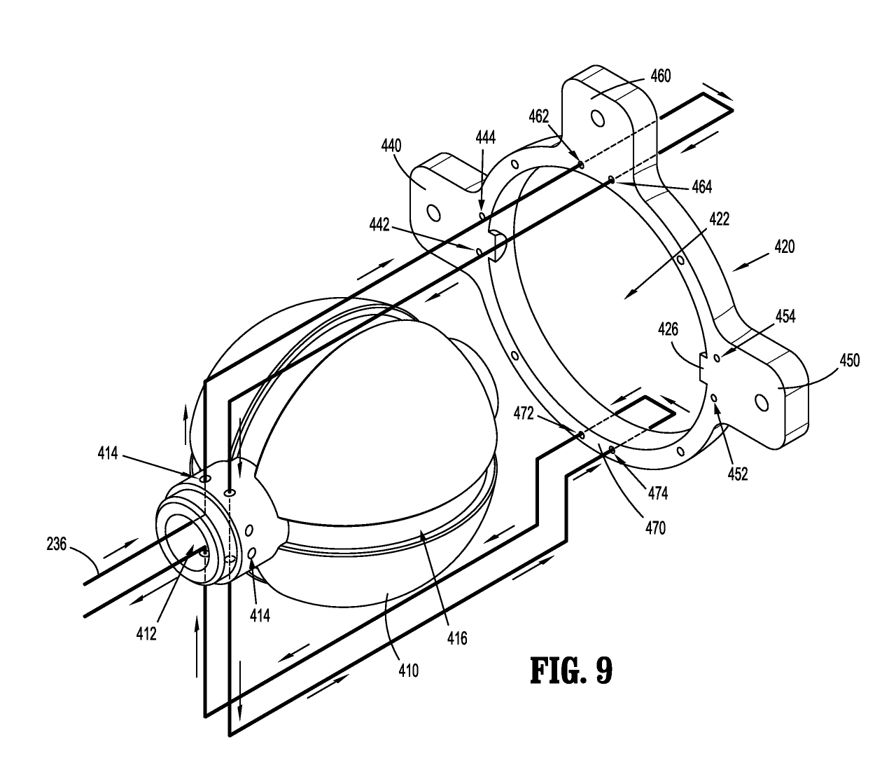

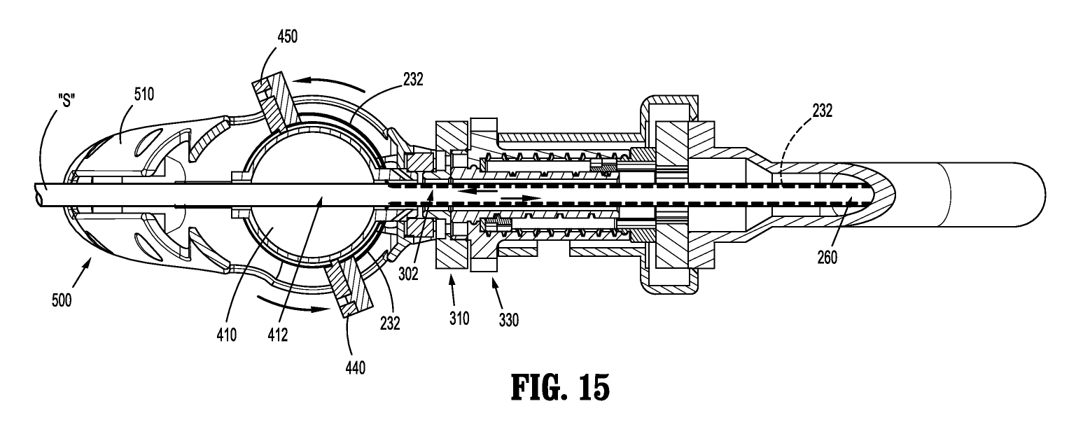

Referring now to FIGS. 9-11 and 13-18, gimbal structure 400 will be described. Gimbal structure 400 includes a spherical member 410 and a ring member 420 disposed about spherical member 410 and rotatable with respect to spherical member 410. Spherical member 410 includes a sphere lumen 412 extending therethrough which is adapted for the reception of flexible surgical instrument “S”. The distal end of sphere lumen 412 is generally aligned with the proximal end of housing lumen 302, as seen in FIG. 15, to allow flexible surgical instrument “S” to pass through sphere lumen 412 and into housing lumen 302. Spherical member 410 is rigidly fixed to housing 300 and includes eight side holes 414 adjacent the distal end for the reception of cables 232 and 236 therethrough. Alternatively, spherical member 410 may include more side holes 414 or less side holes 414 as needed depending on the number of cables to be used and the configuration of the cables with respect to gimbal structure 400. For example, if two additional cables (not shown) were included for use with gimbal structure 400 and cables 232 and 236 and the two new cables (not shown) utilized ferrules instead of looping, it would only be necessary to include four holes. Looping will be described in further detail below with regard to the routing of cables 232 and 236.

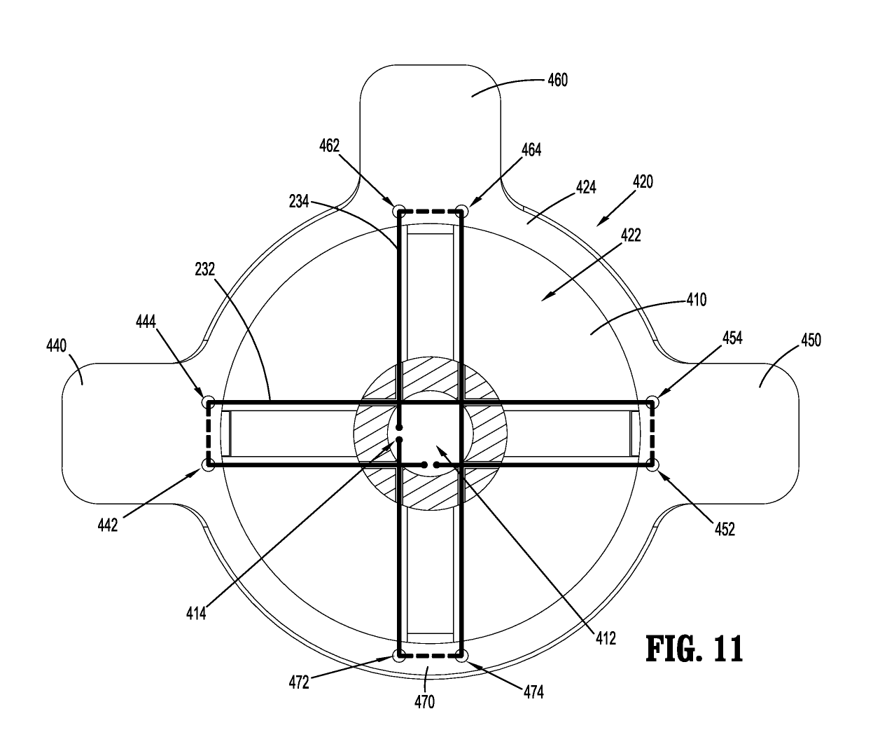

Ring member 420 is configured in a generally circular shape and includes a circular central opening 422 dimensioned for the reception of spherical member 410. In FIG. 11, ring member 420 can be seen in cross section having circular central opening 422 and a ring section 424 with a first flange 440, a second flange 450 and a third flange 460 extending radially therefrom in a generally cross shaped pattern. A fourth side 470 of the cross shaped patter does not include a flange. It is contemplated that a greater or fewer number of flanges may be included as desired and that flanges 440, 450 and 460 may alternatively be disposed in any other configuration to allow for manipulation by a surgeon. Ring section 424 includes a respective pair of holes 442, 444, 452, 454, 462, 464 and 472, 474 extending therethrough for each arm of the cross shaped pattern. Each hole is adapted for the reception of cables 232 and 236 as will be further described below in relation to the cable routing. It is contemplated that holes 442, 444, 452, 454 and 462, 464 may alternatively be disposed on flanges 440, 450 and 460 respectively.

Ring member 420 may further include tabs or protrusions 426 extending radially inward from ring section 424 and adapted to engage a series of channels or grooves 416 defined on spherical member 410. Protrusions 426 may be positioned adjacent flanges 440, 450, 460 and fourth side 470 and may limit the rotation of ring member 420 about spherical member 410 to the pathways defined by grooves 416. It is contemplated that two protrusions 426 may be included adjacent only flanges 440 and 450 or alternatively that four protrusions 426 may be included adjacent flanges 440, 450, 460 and fourth side 470 respectively. It is also contemplated that a greater or fewer number of protrusions 426 may be included and that protrusions 426 may be offset from flanges 440, 450, 460 and fourth side 470. Protrusions 426 and grooves 416 may be used in conjunction with slots 540, 550 and 560 of outer shell 500 to limit the rotation of ring member 420 as will be described in further detail below. Protrusions 426 and grooves 416 are cooperative for permitting movement of ring member 420 along a first axis (i.e. X axis) and along a second axis (i.e. Y axis). Thus, protrusions 426 and grooves 416 are sized such that there is sufficient clearance where they engage each other so that ring member 420 is movable in both the first and the second axes while inhibiting ring member 420 from freely rotating about spherical member 410.

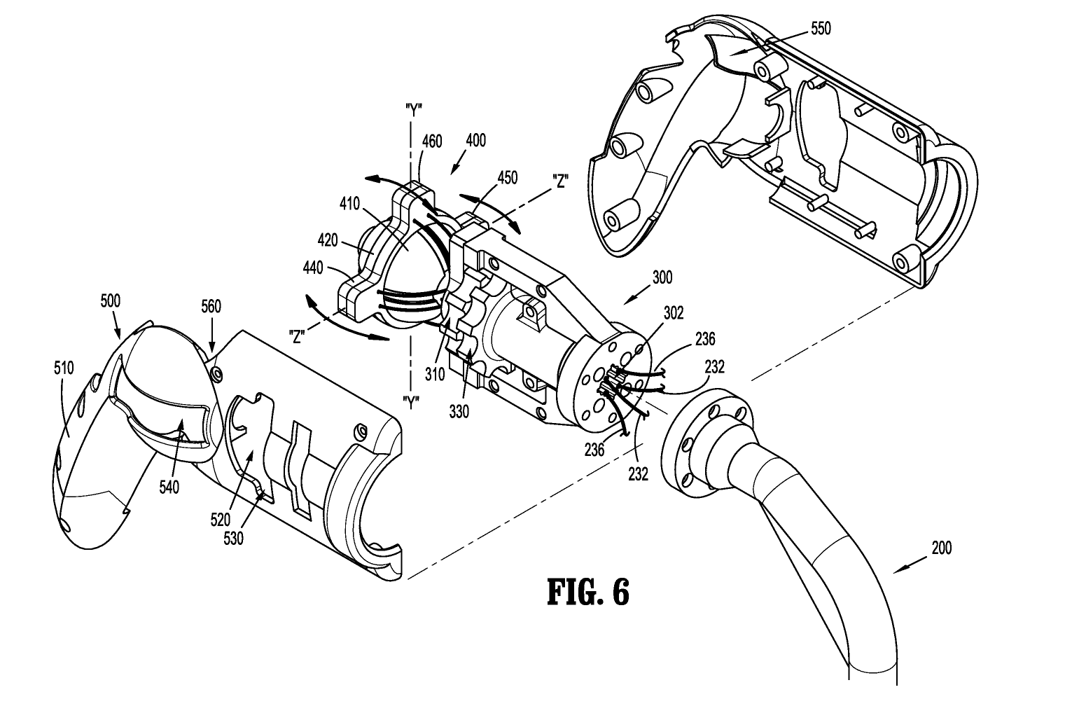

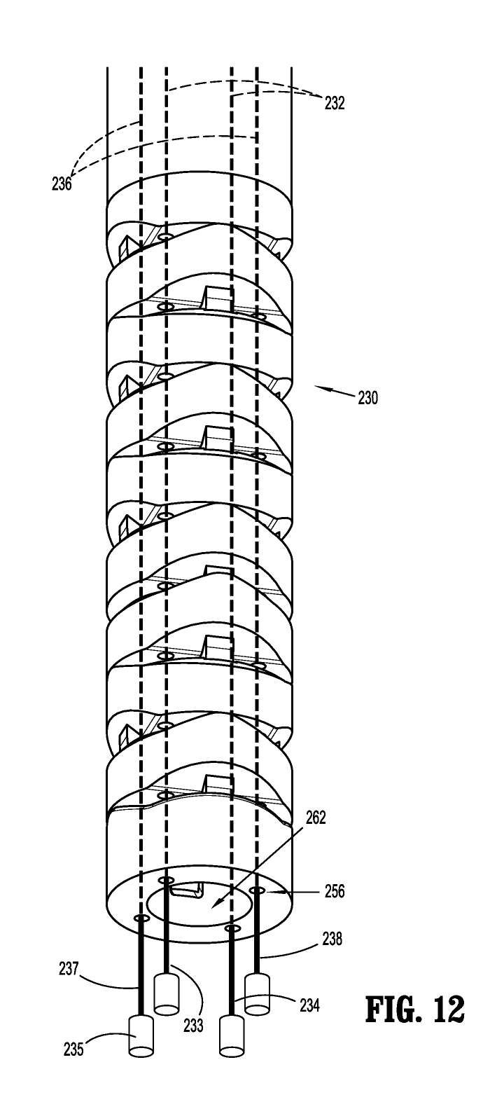

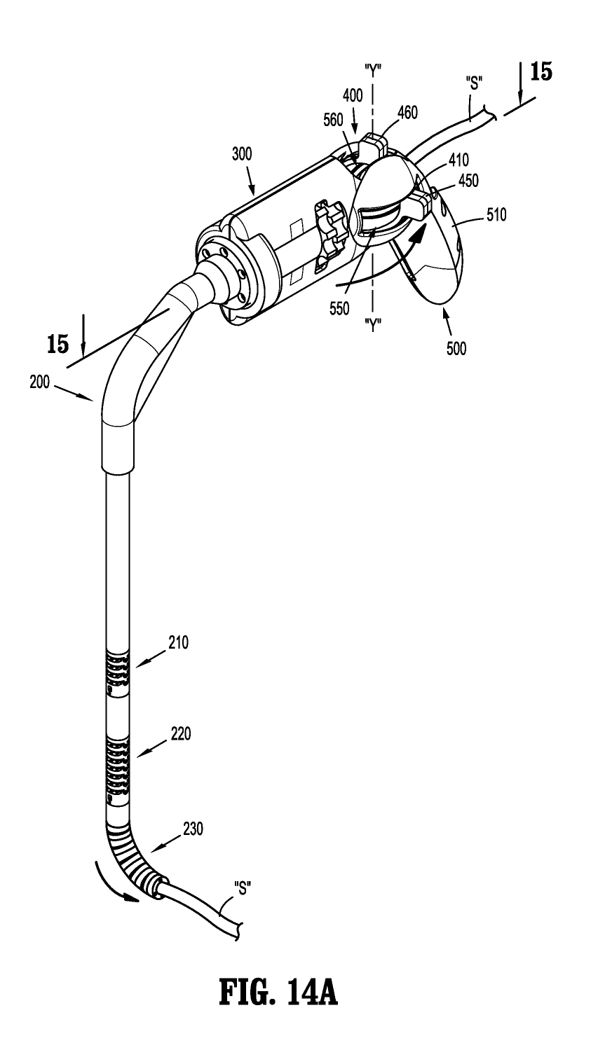

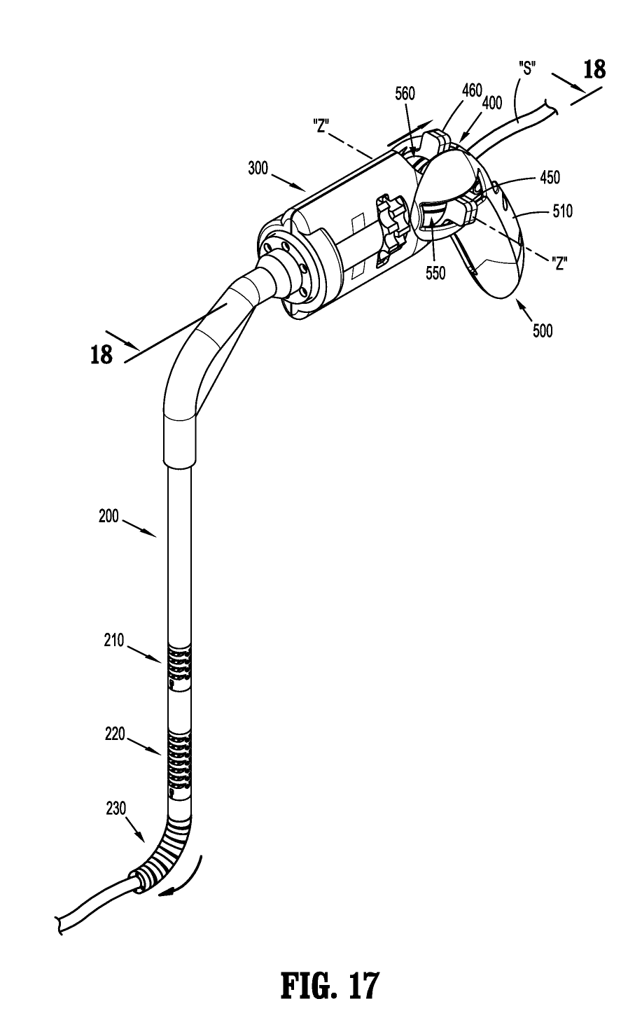

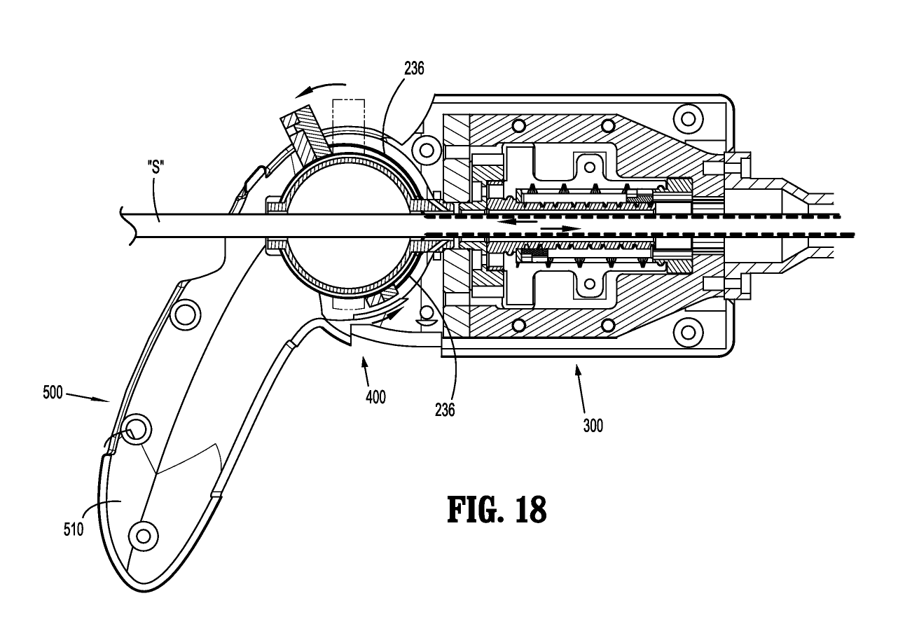

Referring now to FIGS. 5-6, 8-8C, 9 and 11-14, the routing of cables 232 and 236 will be described. As seen in FIG. 12, each of ends 233, 234 and 237, 238 of respective cables 232 and 236 is secured at the distal end of distal section 230 by ferrules 235. Cables 232 and 236 travel along the length of tubular member 200 through channels 270 and through housing 300 to gimbal structure 400, as seen in FIGS. 5 and 6. As seen in FIGS. 9 and 11, once cables 232 and 236 reach gimbal structure 400, cables 232 and 236 exit side holes 414 of spherical member 410 and each loop through a first pair of holes 442, 444 and 462, 464 respectively. After looping through holes 442, 444, and 462, 464 respectively, cables 232 and 236 travel through side holes 414 again and then loop through respective holes 452, 454, and 472, 474, as seen for cable 236 in FIG. 9 and cables 232 and 236, in FIG. 11. After looping through holes 452, 454 and 472, 474 cables 232 and 236 travel through side holes 414 again and return through housing 300 and tubular member 200 to distal section 230 to be secured by ferrules, welding or other methods known in the art. In this way, as seen in FIGS. 14A, 15 and 16, movement of one of flanges 440 or 450 moves one of ends 233 and 234 of cable 232 distally while moving the other of the ends 233 and 234 proximally to manipulate distal section 230 in a first direction. Additionally movement of flange 460 increases moves one of ends 237 and 238 of cable 236 distally while moving the other of the ends 237 and 238 proximally to manipulate distal section 230 in a second different direction, as seen if FIGS. 17 and 18. It is contemplated that distal section 230 may be manipulated in multiple directions by moving flanges 440, 450 and 460 where, for example, movement of one of flanges 440 and 450 in conjunction with flange 460 manipulates distal section 230 in a third direction or fourth direction. It is contemplated that cables 232 and 236 may alternatively be attached to ring member 420 by a ferrule (not shown) rather than looping through holes 442, 444, 452, 454, 462, 464 and 472, 474 as disclosed above and that there may be four cables instead of two.

Referring now to FIGS. 1-4 and 6, outer shell 500 can be seen. Outer shell 500 is disposed around both housing 300 and gimbal structure 400 and includes a handle 510, openings 520 and 530 for the reception of control members 310 and 330 respectively and slots 540, 550 and 560 for the reception of flanges 440, 450 and 460 respectively. Handle 510 also defines an open section 512 to provide flexible surgical instrument “S” with access to sphere lumen 412. Openings 520 and 530 are dimensioned to provide a surgeon with access to control members 310 and 330. Slots 540, 550 and 560 are dimensioned to limit the rotation of ring member 420 by limiting the rotational movement of flanges 440, 450 and 460 to respective paths defined by slots 540, 550 and 560. Slots 540 and 550 allow ring member 420 to rotate around a first vertical axis “Y” which is perpendicular to a longitudinal axis of sphere lumen 412 while slot 560 allows ring member 420 to rotate around a second horizontal axis “Z” which is perpendicular to both first vertical axis “Y” and the longitudinal axis of sphere lumen 412. In this way rotation of ring member 420 is limited to two axes to prevent tangling of cables 232 and to provide a surgeon with an indicator of the relative position of distal section 230 by reducing the possible directions of articulation. As discussed above, slots 540, 550 and 560 may be used in conjunction with grooves 416 and protrusions 426 to limit the rotational movement of ring member 420 where grooves 416 and protrusions 426 assist slots 540, 550 and 560 in allowing rotation only about the “Y” and “Z” axes. It is contemplated that slots 540, 550 and 560 and grooves 416 and protrusions 426 may alternatively be dimensioned to allow ring member 420 to rotate in more than two axes of movement or in other different axes of movement.

During use, referring now to FIGS. 14A-18, tubular member 200 is inserted into a surgical site through a natural orifice or an incision in tissue. Natural orifices may include, for example, the mouth, anus, vagina, etc. Tubular member 200 may also be inserted into the surgical site through a surgical access portal. A flexible surgical instrument “S” is then inserted through open section 512, sphere lumen 412, housing lumen 302 and flexible lumen 260 and into the surgical site. Once flexible surgical instrument “S” is in position within the surgical site a surgeon may manipulate control member 310 by rotating knob 312 to increase the tension on one of cables 212 and 214 while decreasing the tension on the other of cables 212 and 214 to articulate flexible links 240 of proximal section 210. The surgeon may also manipulate control member 330 by rotating knob 332 to increase the tension on one of cables 222 and 224 while decreasing the tension on the other of cables 222 and 224 to articulate flexible links 240 of central section 220. This provides the surgeon with coarse control of tubular member 200 and thus flexible surgical instrument “S” in the surgical site. Once flexible surgical instrument “S” is positioned in the desired region of the surgical site the surgeon then utilizes gimbal structure 400 to provide for fine control of distal section 230 of tubular member 200 and thus flexible surgical instrument “S”. The surgeon manipulates first flange 440 or second flange 450 to rotate ring member 420 about first vertical axis “Y” which also rotates the other of the flanges 440 and 450 about first vertical axis “Y”. Rotating ring member 420 about first vertical axis “Y” causes one of ends 233 and 234 of cable 232 to be moved proximally while the other of ends 233 and 234 is moved distally to articulate distal section 230 in the direction of the proximally moving end. The surgeon may also manipulate third flange 460 to rotate ring member 420 about second horizontal axis “Z” which also rotates fourth side 470 about second horizontal axis “Z”. Rotating ring member 420 about second horizontal axis “Z” causes one of ends 237 and 238 of cable 236 to be moved proximally while the other of ends 237 and 238 is moved distally to articulate distal section 230 in the direction of the of the proximally moving end. In this way the surgeon may easily and effectively articulate distal section 230 of tubular member 200 in any direction as necessary during surgery while still maintaining the fine control that is necessary for a surgical operation.

Although the present disclosure has been described with respect to preferred embodiments, it will be readily apparent, to those having ordinary skill in the art that changes and modifications may be made thereto without departing from the spirit or scope of the subject apparatus.

1. A surgical articulation apparatus for articulation of laparoscopic instruments comprising:
a housing, the housing defining a housing lumen therethrough for the reception of a surgical instrument;
a tubular member extending distally from the housing, the tubular member defining a flexible lumen therethrough for the reception of the flexible surgical instrument;
a spherical member attached to a proximal end of the housing, the spherical member defining a sphere lumen therethrough for the reception of the flexible surgical instrument, the lumen defining a longitudinal axis;
a ring member disposed about the spherical member and rotatable with respect to the spherical member; and
a cable attached to the tubular member and extending through the housing to the ring member, the cable being disposed in mechanical communication with the ring member such that the tubular member is articulated by the cable upon rotation of the ring member about the spherical member.

2. The surgical articulation apparatus of claim 1, wherein the ring member includes a plurality of openings therethrough, the cable being inserted through at least one of the plurality of openings.

3. The surgical articulation apparatus of claim 2, wherein the cable defines two ends, each end being attached to a distal section of the tubular member.

4. The surgical articulation apparatus of claim 1, wherein the ring member further includes a plurality of flanges disposed on its surface, the flanges actuatable to rotate the ring member about the spherical member.

5. The surgical articulation apparatus of claim 4, further including an outer shell disposed around the housing, the ring member and the spherical member.

6. The surgical articulation apparatus of claim 5, wherein the outer shell includes a handle portion, the handle portion defining a plurality of slots thereon for the reception of the plurality of flanges therethrough.

7. The surgical articulation apparatus of claim 6, wherein the plurality of slots are dimensioned to allow at least one the flanges to rotate about a first axis perpendicular to the longitudinal axis and another of the flanges to rotate about a second axis perpendicular to both the first axis and the longitudinal axis.

8. The surgical articulation apparatus of claim 1, wherein the tubular member includes a series of flexible elements.

9. A method of using a surgical articulation apparatus comprising the steps of:
providing a surgical articulation apparatus for articulation of laparoscopic instruments comprising:
a housing, the housing defining a housing lumen therethrough for the reception of a flexible surgical instrument;
a tubular member extending distally from the housing, the tubular member defining a flexible lumen therethrough for the reception of the flexible surgical instrument;
a spherical member attached to a proximal end of the housing, the spherical member defining a sphere lumen therethrough for the reception of the flexible surgical instrument, the sphere lumen defining a longitudinal axis;
a ring member disposed about the spherical member and rotatable about the spherical member; and
a cable attached to the tubular member and extending through the housing to the ring member, the cable being disposed in mechanical communication with the ring member such that the tubular member is articulated by the cable upon rotation of the ring member about the spherical member;
inserting the tubular member into a surgical site;
inserting the flexible surgical instrument through the sphere lumen, housing lumen and flexible lumen and into the surgical site;
rotating the ring member about a first axis to manipulate the tubular member in a first direction; and
rotating the ring member about a second axis to manipulate the tubular member in a second direction.

10. The method of claim 9, wherein the ring member further includes a plurality of flanges disposed on its surface.

11. The method of claim 10, wherein the step of rotating the ring member about the first axis includes manipulating a first of the plurality of flanges about the first axis, the first axis being perpendicular to the longitudinal axis of the sphere lumen.

12. The method of claim 10, wherein the step of rotating the ring member about the second axis includes manipulating a second of the plurality of the flanges about the second axis, the second axis being perpendicular to the longitudinal axis of the sphere lumen and perpendicular to the first axis.

13. The method of claim 10, wherein the plurality of flanges define a generally cross shaped pattern with flanges on three of the four sides.

14. The method of claim 9, wherein rotation of the ring member translates a first end of the cable in one of a distal or proximal direction and a second end of the cable in the other of the distal or proximal direction to articulate the tubular member.

15. The method of claim 14, wherein the first and second ends of the cable are attached to a distal section of the tubular member, the cable being looped through at least one hole in the ring member.

16. The method of claim 15, wherein the cable is looped through two holes in each of a first and second sides of the ring member before returning to the distal section of the tubular member.

17. The method of claim 16, further including a second cable attached to the distal section of the tubular member and looping through two holes in each of a third and fourth sides of the ring member before returning to the distal section of the tubular member.

18. The method of claim 9, wherein the tubular member further includes flexible links.

19. The method of claim 18, wherein upon movement of the cable in a distal or proximal direction, the flexible links allow the tubular member to bend.

20. A surgical articulation apparatus for articulation of laparoscopic instruments comprising:
a housing, the housing defining a housing lumen therethrough for the reception of a flexible surgical instrument;
an tubular member extending distally from the housing, the tubular member defining a flexible lumen therethrough for the reception of the flexible surgical instrument and a series of flexible linked sections adapted to allow the tubular member to flex;
a gimbal attached to a proximal end of the housing, the gimbal including:
a spherical member defining a lumen therethrough for the reception of the flexible surgical instrument; and
a rotating member disposed around the spherical member and rotatable with respect to the spherical member, the rotating member defining a plurality of holes therethrough; and
at least one wire attached to a distal end of the tubular member and extending through the housing to the gimbal, the wire looping through at least two of the plurality of holes and further extending from the gimbal back through the housing to further attach to the distal end of the tubular member, the at least one wire adapted to flex the tubular member upon rotation of the rotating member.
